(19) United States
(12) Patent Application Publication
(10) Pub. No.: US 2026/0032795 A1
(43) Pub. Date: Jan. 29, 2026

(54) SYSTEM FOR CONTROLLING UV-C LED

(71) Applicant: AeroClenz, Inc., Bonita Springs, FL (US)

(72) Inventor: Matthew A. Saberton, Bonita Springs, FL (US)

(73) Assignee: AeroClenz, Inc., Bonita Springs, FL (US)

(21) Appl. No.: 18/784,845

(22) Filed: Jul. 25, 2024

(51) Int. Cl. H05B 47/115; A61L 2/10; H05B 45/10; H05B 45/325

(52) U.S. Cl. CPC H05B 47/115; A61L 2/10; H05B 45/10; H05B 45/325; A61L 2202/11

(57) ABSTRACT

A system for controlling a UV-C LED comprising a biological characteristic and/or recognition presence sensor, configured to measure the distance between the sensor and a living organism, and a controller programmed to receive the measured distance and calculate an adjusted power level for the UV-C LED based on the measured distance to maintain a predetermined safe UV-C irradiance level. The controller employs a dynamic adjustment algorithm to determine the adjusted power level of the UV-C LED to ensure that the UV-C irradiance remains below a maximum allowable exposure level as the living organism's distance changes. The dynamic adjustment algorithm comprises a representing a safety distance within which the UV-C LED should be deactivated to ensure safety. The controller may utilize the micro-Doppler effect to allow for a precise and reliable means for detecting and analyzing micro-scale movements that may be associated with breathing and heartbeat.

### FIELD OF INVENTION

The present invention is a system to control a UV-C LED to maintain a safe ultraviolet (UV) irradiance exposure.

### BACKGROUND

In today's world, maintaining a clean and sanitized environment is paramount to safeguarding public health and preventing the spread of infectious diseases. With the constant threat of pathogens lurking on surfaces and in the air, the need for effective disinfection methods has never been more pressing. Traditional disinfection techniques often involve the use of chemicals, which can be harsh, leave behind residues, and contribute to environmental pollution.

Ultraviolet (UV) irradiance is electromagnetic irradiance with a wavelength falling between 100 nm and 400 nm. It is split into four spectral areas. One of those spectral areas, UV-C, rests between 200 to 280 nm. This range is lethal for microorganisms, as it is strongly absorbed by the nucleic acids of a microorganism. This allows it to be used for disinfectant purposes, making UV-C light a viable alternative to current disinfecting methods.

### SUMMARY OF THE INVENTION

The present disclosure relates to a system for controlling a UV-C light emitting diode (LED). In accordance with an embodiment, the system includes a mmWave sensor configured to measure the distance between the sensor and a living organism; and a controller programmed to receive the measured distance and calculate an adjusted power level for the UV-C LED based on the measured distance to maintain a predetermined safe UV-C irradiance level. In other embodiments, the system includes, instead of or in addition to a mmWave sensor, another type of sensor (cameras, ultrasound sensors, passive infrared sensors (PIR), light detection and raging sensors (LIDAR), etc.) that detects presence and/or distance between the sensor and the living organism to provide an adjusted power level for the UV-C LED based on the measured distance to maintain a predetermined safe UV-C irradiance level.

In one embodiment, the dynamic adjustment algorithm calculates a proximity ratio based on the measured distance and a predefined safety parameter, and adjusts the power level of the UV-C LED according to the proximity ratio.

In one embodiment, the predefined safety parameter is 300 mm, representing a safety distance within which the UV-C LED should be deactivated to ensure safety.

In one embodiment, the system further includes a fail-safe mechanism that triggers an immediate shutdown of the UV-C LED if anomalies or errors are detected in distance measurements or power control processes.

In one embodiment, the controller uses pulse-width modulation (PWM) techniques to adjust the power supplied to the UV-C LED.

### BRIEF DESCRIPTION OF THE DRAWINGS

FIGS. 1A and 1B illustrate an example system for controlling a UV-C light emitting diode (LED).

FIG. 2 illustrates a block diagram of an example system for controlling a UV-C LED.

FIG. 3 illustrates a flow chart for a dynamic adjustment algorithm.

FIG. 4 illustrates a flow chart for steps on moving the actinic dosage limit.

FIG. 5 illustrates a flow chart of an example of a possible mathematical algorithm for updating the position of the actinic dosage limit.

FIG. 6 illustrates a flow chart of an example of a mathematical algorithm that dynamically adjusts the power level for the UV-C LED.

FIG. 7 illustrates a flow chart on an example of incorporating the inverse square law.

FIG. 8 shows an example system for controlling a multiple UV-C LEDs.

### DETAILED DESCRIPTION

A current problem in using ultraviolet (UV) light, specifically UV-C, to disinfect an area is avoiding exposing unintended living organisms (e.g., humans) to harmful UV-C irradiance levels while also maintaining enough power to still disinfect by killing intended living organisms (e.g., microorganisms). The term actinic dosage limit refers to the maximum allowable exposure to actinic (ultraviolet) radiation that is considered safe for human skin and eyes. Actinic radiation, particularly in the UV spectrum, can cause harmful biological effects, including skin burns, eye damage, and an increased risk of skin cancer. The limits are established to minimize these risks and are based on scientific research and safety guidelines. Different organizations and standards bodies, such as the International Commission on Non-Ionizing Radiation Protection (ICNIRP) and the American Conference of Governmental Industrial Hygienists (ACGIH), provide guidelines and exposure limits for UV radiation. These limits are typically specified in terms of irradiance (power per unit area) and exposure duration. For example, the ICNIRP guidelines for UV radiation exposure include limits on effective irradiance (measured in watts per square meter, W/m²) over an 8-hour workday, with specific limits for different UV wavelength ranges. The International Electrotechnical Commission (IEC) uses the ICNIRP guidelines along with data from experimental studies to determine exposure limits for individuals in the vicinity of lamp and lamp systems, which can be found in Chapter 4 of IEC 62471:2006. Similarly, the ACGIH TLVs (Threshold Limit Values) provide limits for UV exposure to prevent acute and chronic health effects.

The system described herein uses presence (e.g., mmWave) sensors to provide real-time feedback to indicate the presence and physiological activity of individuals in a UV-C system's vicinity. This allows a processor to quickly determine the actinic dosage limit, such that a maximum power can be applied to the UV-C light emitting diode (LED) without overexposing any unintended living organism (hereinafter referred to simply as “living organism”) detected by the UV-C system. A goal of the invention described herein is to maintain irradiation to living organisms under DIBEL (Direct Irradiance Below Exposure Limits), a term used in the context of light and radiation exposure, specifically indicating that the level of irradiance (the power of electromagnetic radiation per unit area) is below the established safety exposure limits for humans or other living organisms. The sensor or sensors constantly measures the distance to allow the processor to adjust the actinic dosage limit as the living organism moves closer to or farther from the UV-C system. A safety parameter may be included in the calculation of the actinic dosage limit to create a safety threshold between safe and harmful UV-C exposure. The safety parameter may adjust with the distance of the living organism.

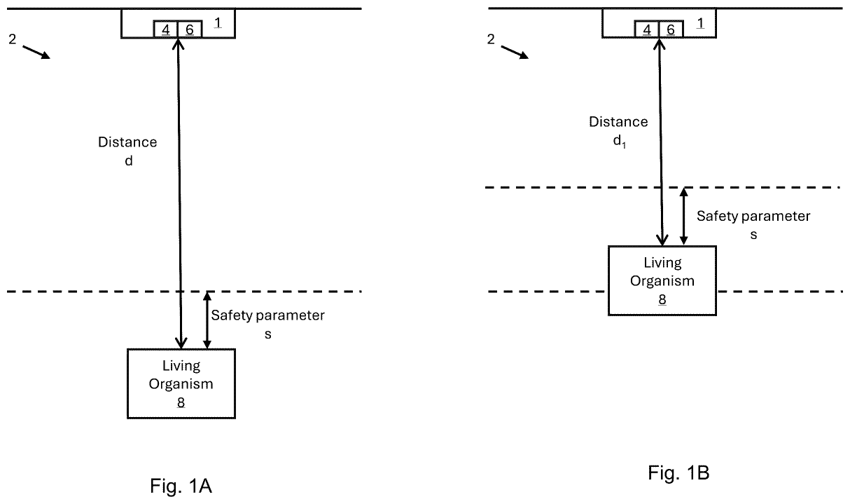

FIGS. 1A and 1B show an example of a system Error! Reference source not found. for controlling a UV-C LED for disinfecting purposes. The system Error! Reference source not found. can work in both indoor and outdoor environments. A non-limiting example may be in an aircraft. To best explain the functionality of the system, it will be explained in the context of an aircraft Error! Reference source not found. The system Error! Reference source not found. containing a UV-C LED Error! Reference source not found. and a presence sensor Error! Reference source not found. may be installed within a confined compartment of an aircraft, such as a lavatory, gallery, or cockpit. The UV-C LED Error! Reference source not found. contained inside of the system Error! Reference source not found. emits ultraviolet irradiance at 200 to 280 nm. Exposure to high concentrations of irradiance can be harmful to living organisms. Therefore, the system Error! Reference source not found. aims to solve this problem by limiting the amount of irradiance to living organisms, based on their distance from the system Error! Reference source not found. Examples of presence sensors include millimeter wave (mmWave) sensors (a type of radar technology that operates in the millimeter-wave frequency range, typically between 30 GHz and 300 GHz) but may also include cameras, ultrasound sensors, passive infrared sensors (PIR), light detection and raging sensors (LIDAR), etc. In the present disclosure, we refer to the presence sensor 6 as a mmWave sensor but, in some embodiments, the disclosed systems and techniques may be executed using other types of presence sensors as listed above.

In the example of an aircraft Error! Reference source not found., a person Error! Reference source not found. (or alternatively another living organism, such as pets) may be sitting in a seat inside the aircraft. For sanitation purposes, the airline may want to disinfect the aircraft while passengers are still inside. Existing technologies utilizing UV-C disinfectants run the risk of overexposing a passenger to harmful ultraviolet irradiance. In the system Error! Reference source not found., a mmWave sensor Error! Reference source not found. detects the person 8 and calculates a distance d. The system Error! Reference source not found. then creates a safety threshold defined as a safety parameter s. This safety parameter is the distance at which the UV-C LED Error! Reference source not found. should be turned off to ensure the person Error! Reference source not found. remains within a safe exposure limit. An example distance of the safety parameter s may be 300 mm. If a processor used to calculate the safety parameter s is slow, a larger safety parameter s may need to be chosen. The processor must be able to recalculate the safety parameter s in the event the person Error! Reference source not found. moves closer to the system Error! Reference source not found. fast enough to ensure the safety of person 8.

As demonstrated in FIG. 1A, the system Error! Reference source not found. calculates a first value for the safety parameter s for the person Error! Reference source not found. in a first location. In FIG. 1B, the person Error! Reference source not found. has moved closer to the system Error! Reference source not found. As such, the person Error! Reference source not found. has now exceeded the previously calculated safety threshold. A new safety parameter must be calculated based on the new distance di between the person Error! Reference source not found. and the system Error! Reference source not found.

In addition to needing to adjust the safety parameter s, the system Error! Reference source not found. also may adjust the amount of power flowing into the UV-C LED Error! Reference source not found., limiting ultraviolet irradiance being emitted. The adjusted ultraviolet irradiance may be based on a calculated actinic dosage limit for the distance d of the living organism Error! Reference source not found., which signifies the maximum permissible UV-C dosage for safe exposure. Exceeding the actinic dosage limit can result in harmful exposure. The system Error! Reference source not found. may constantly adjust the ultraviolet irradiance based on the actinic dosage limit to allow the maximum permissible UV-C dosage while maintaining a safe exposure.

If the mmWave sensor Error! Reference source not found. detects more than one living organism Error! Reference source not found., the calculations may be performed based on the closest living organism Error! Reference source not found. to the system Error! Reference source not found. This ensures that no living organism Error! Reference source not found. experiences a harmful exposure to ultraviolet irradiance. As the mmWave sensor Error! Reference source not found. is constantly scanning the area Error! Reference source not found., the system Error! Reference source not found. will be able to detect any living organism Error! Reference source not found. that enters the scanning radius. If a farther organism Error! Reference source not found. moves closer to the system Error! Reference source not found., the actinic dosage limit calculations will be performed with respect to the now closer living organism Error! Reference source not found.

The system Error! Reference source not found. uses a mmWave sensor Error! Reference source not found. coupled with the micro-Doppler effect to detect living organisms Error! Reference source not found. The mmWave sensor Error! Reference source not found. utilizes the time of flight (TOF) principle to calculate the distance d of the person Error! Reference source not found. Signals from mmWave sensors Error! Reference source not found. can easily penetrate obstacles or surfaces between the sensor and the person Error! Reference source not found., allowing the mmWave sensor Error! Reference source not found. to detect and track individuals, even when partially or fully obstructed by objects like furniture, walls, or curtains.

The micro-Doppler effect refers to the phenomenon of detecting and analyzing the Doppler shift in the radar return signal caused by the motion of individual components within a target. The micro-Doppler effect in mmWave sensing refers to small frequency variations in reflected mmWave signals caused by micro-scale movements of objects within the sensor's field of view. These movements include physiological activities such as breathing or heartbeat. Signal processing algorithms, including but not limited to time-frequency analysis and pattern recognition, can be used to analyze the reflected mmWave signals. By analyzing the variations in frequency over time, these algorithms can accurately identify and differentiate micro-Doppler signatures associated with physiological activities from other sources of noise or interference.

Techniques such as, but not limited to, Fourier analysis or wavelet transforms can be applied to decompose the mmWave signal into its frequency components and track the variations in those components over time. These techniques allow for the extraction of the micro-Doppler signatures related to the physiological activities of interest. Pattern recognition algorithms can then be utilized to classify and distinguish these micro-Doppler signatures. Machine learning algorithms, such as support vector machines (SVM), can be trained on a dataset of known micro-Doppler signatures to accurately identify and classify the different physiological activities of interest. The processor can receive information about the frequency shifts and patterns associated with specific physiological activities.

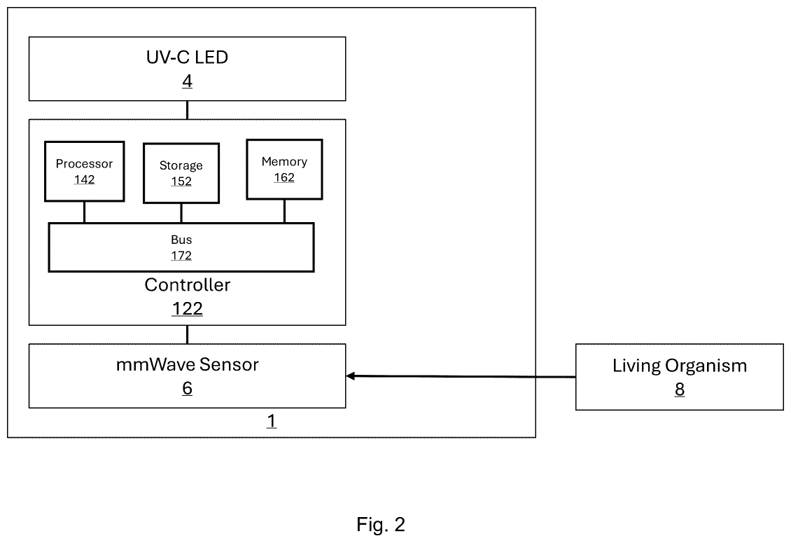

FIG. 2 illustrates a block diagram of an exemplary system Error! Reference source not found. for controlling a UV-C LED. The system Error! Reference source not found. includes a UV-C LED Error! Reference source not found., a controller Error! Reference source not found., and a mmWave sensor Error! Reference source not found. The controller Error! Reference source not found. contains a processor Error! Reference source not found., a storage Error! Reference source not found., and a memory Error! Reference source not found., all operably connected by a bus Error! Reference source not found. The mmWave sensor Error! Reference source not found. detects the distance of a living organism Error! Reference source not found., and relays that information to the controller Error! Reference source not found. to calculate the appropriate maximum allowable UV-C dosage. The controller Error! Reference source not found. continuously reads the distance measurements provided by the mmWave sensor Error! Reference source not found. These measurements provide precise information about the spatial separation between the sensor Error! Reference source not found. and the living organism Error! Reference source not found.

The processor Error! Reference source not found. can be a variety of various processors including dual microprocessor and other multi-processor architectures. The memory Error! Reference source not found. can include volatile memory or non-volatile memory. The non-volatile memory can include, but is not limited to, ROM, PROM, EPROM, EEPROM, and the like. Volatile memory can include, for example, RAM, synchronous RAM (SRAM), dynamic RAM (DRAM), synchronous DRAM (SDRAM), double data rate SDRAM (DDR SDRAM), and direct RAM bus RAM (DRRAM). The storage Error! Reference source not found. may be operably connected to the processor Error! Reference source not found. via the bus Error! Reference source not found. The storage Error! Reference source not found. can include, but is not limited to, devices like a magnetic disk drive, a solid-state disk drive, a flash memory card, or a memory stick. The memory Error! Reference source not found. can store processes or data.

The bus Error! Reference source not found. can be a single internal bus interconnect architecture or other bus or mesh architectures. While a single bus is illustrated, it is to be appreciated that the battery management system 2 may communicate with various devices, logics, and peripherals using other buses that are not illustrated (e.g., PCIE, SATA, InfiniBand, 1394, USB, Ethernet). The bus Error! Reference source not found. can be of a variety of types including, but not limited to, a memory bus or memory controller, a peripheral bus or external bus, a crossbar switch, or a local bus. The local bus can be of varieties including, but not limited to, an industrial standard architecture (ISA) bus, a microchannel architecture (MCA) bus, an extended ISA (EISA) bus, a peripheral component interconnect (PCI) bus, a universal serial (USB) bus, and a small computer systems interface (SCSI) bus.

The controller begins by initializing the mmWave sensor Error! Reference source not found. and UV-C LED Error! Reference source not found. module, establishing the necessary communication and control interfaces. The controller Error! Reference source not found. adjusts the power of the UV-C LED Error! Reference source not found. to ensure that the maximum allowable UV-C dosage remains aligned with the living organism's Error! Reference source not found. changing distance. The controller Error! Reference source not found. defines a variable representing an actinic dosage limit, which signifies the maximum permissible UV-C dosage for safe exposure based on the distance of a living organism.

Based on the distance measurement, the controller Error! Reference source not found. applies a dynamic adjustment algorithm to calculate the optimal power level for the UV-C LED Error! Reference source not found. This algorithm considers the current distance, a safety parameter which indicates the distance at which the UV-C LED should be turned off to ensure the living organism 8 remains within a safe exposure limit, and the calculated actinic dosage limit. A non-limiting example of an adequate safety parameter may be 300 mm. The distance range created by the safety parameter in respect to the living organism Error! Reference source not found. is dependent on the processor used in the controller Error! Reference source not found. A slower processor would need a larger safety parameter to ensure that the power to the UV-C LED Error! Reference source not found. is reduced in time before the living organism Error! Reference source not found. crosses the actinic dosage limit.

By using pulse-width modulation (PWM) or similar techniques, the controller Error! Reference source not found. modulates the power supplied to the UV-C LED Error! Reference source not found. module. The power level is adjusted in accordance with the calculated value from the dynamic adjustment algorithm, ensuring precise control over the UV-C irradiance.

As the living organism Error! Reference source not found. moves closer to or farther away from the UV-C LED Error! Reference source not found., the controller Error! Reference source not found. updates the actinic dosage limit dynamically. The limit's position adjusts with the living organism's Error! Reference source not found. movement, while continuously keeping a distance from the living organism equal to the safety parameter. This responsive behavior guarantees that the UV-C exposure remains within safe limits, accommodating changes in the living organism's Error! Reference source not found. position.

The controller Error! Reference source not found. continually monitors the distance measurements and updates the power level of the UV-C LED Error! Reference source not found. in real-time. This ensures that the UV-C exposure remains below the adjusted actinic dosage limit, preventing any possibility of overexposure.

In the flow diagrams, blocks denote “processing blocks” that may be implemented with logic. The processing blocks may represent a method step or an apparatus element for performing the method step. The flow diagrams do not depict syntax for any particular programming language, methodology, or style (e.g., procedural, object-oriented). Rather, the flow diagrams illustrate functional information one skilled in the art may employ to develop logic to perform the illustrated processing. It will be appreciated that in some examples, program elements like temporary variables, routine loops, and so on, are not shown. It will be further appreciated that electronic and software applications may involve dynamic and flexible processes so that the illustrated blocks can be performed in other sequences that are different from those shown or that blocks may be combined or separated into multiple components. It will be appreciated that the processes may be implemented using various programming approaches like machine language, procedural, object oriented or artificial intelligence techniques.

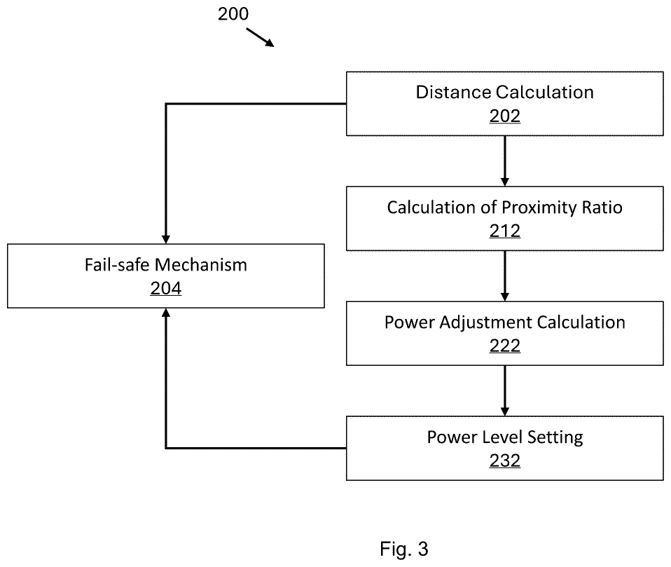

FIG. 3 illustrates a flow diagram of one example for a dynamic adjustment algorithm Error! Reference source not found. At step Error! Reference source not found., a controller Error! Reference source not found. (FIG. 2) measures the distance between a living organism Error! Reference source not found. (FIG. 1A) and an mmWave sensor Error! Reference source not found. (FIG. 1A) using time of flight (TOF) principles. This distance is denotated in future calculations as “d.” The controller Error! Reference source not found. compares the measured value to a stored detects an error in the distance measurement, such as the distance recorded being too high, the controller falls to the fail-safe mechanism at step Error! Reference source not found.

At step Error! Reference source not found., the controller triggers an immediate shutdown of the UV-C LED Error! Reference source not found. (FIG. 1A) to mitigate risks of overexposure. At step Error! Reference source not found., a proximity ratio “r” is calculated. This ratio is between the current distance “d” and a desired 300 mm safety parameter “s.” It can be expressed as r=d/s. The safety parameter “s” may be a predefined constant. The safety parameter determines the distance at which the UV-C LED Error! Reference source not found. should be turned off to ensure the living organism remains within a safe exposure limit. Depending on the processing speed of the controller Error! Reference source not found. (FIG. 2), a different safety parameter distance may need to be used. The distance 300 mm is an example safety parameter, although the safety parameter may be higher if the processor Error! Reference source not found. (FIG. 2) is slower.

At step Error! Reference source not found., the power adjustment is calculated. The power adjustment factor “p” is determined based on the proximity ratio “r” using a mathematical function or a corresponding lookup table specific to the UV-C LED module. The power adjustment factor determines the appropriate power level required to maintain the desired UV-C irradiance for the given proximity ratio and is expressed as: p=ƒ(r). The function ƒ(r) can be defined based on the specific characteristics and requirements of the UV-C LED Error! Reference source not found. (FIG. 1A) module. It may involve mathematical equations, interpolation, or other suitable methods to map the proximity ratio to the corresponding power adjustment factor.

At step Error! Reference source not found., the controller adjusts the power supplied to the UV-C LED Error! Reference source not found. using pulse-width modulation (PWM) or other appropriate techniques. The power level is set as a proportion of the maximum power output of the UV-C LED Error! Reference source not found. module, determined by multiplying the power adjustment factor “p” with the maximum power level and can be expressed as: Power Level=p*Max Power Output. By calculating the proximity ratio and determining the power adjustment factor based on this ratio, the controller Error! Reference source not found. (FIG. 2) modulates the power supplied to the UV-C LED Error! Reference source not found. module. This ensures that the UV-C irradiance is adjusted in accordance with the living organism's Error! Reference source not found. proximity, maintaining a safe and effective disinfection environment. If an error occurs while the controller Error! Reference source not found. attempts to set the power level at step Error! Reference source not found., the controller Error! Reference source not found. falls back to the fail-safe mechanism in step Error! Reference source not found. and shuts down the UV-C LED Error! Reference source not found. Potential errors may include, but are not limited to, faulty data being used, the power adjustment calculation exceeding a safe threshold, or the power level set does not equal the power level calculated at step Error! Reference source not found.

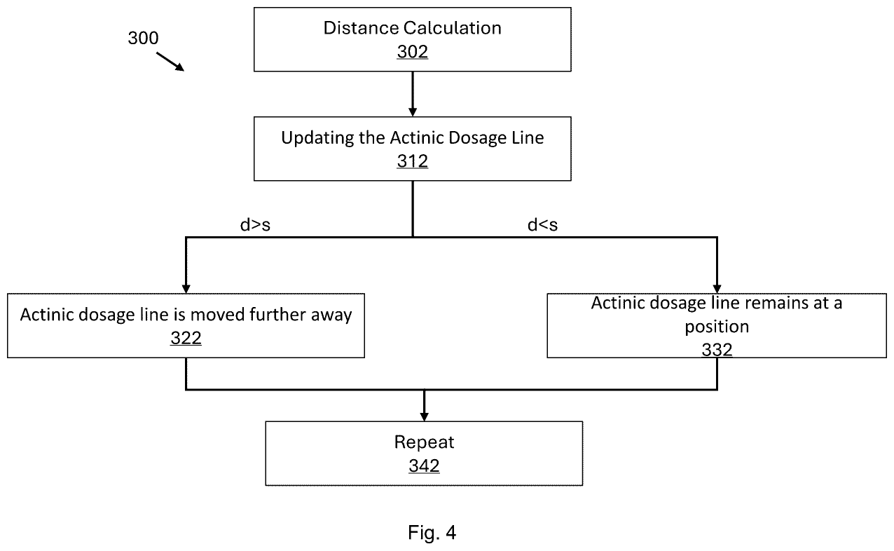

FIG. 4 illustrates a flow diagram for an example of moving an actinic dosage limit Error! Reference source not found. A controller Error! Reference source not found. (FIG. 2) dynamically updates the position of the actinic dosage limit as a living organism's Error! Reference source not found. (FIG. 1A) proximity to the UV-C LED Error! Reference source not found. (FIG. 1) changes. The line's movement is determined by a mathematical algorithm that continuously aligns the actinic dosage limit with the desired safety parameter. At step Error! Reference source not found., the controller Error! Reference source not found. measures the distance between the living organism Error! Reference source not found. and an mmWave sensor Error! Reference source not found. (FIG. 1A) utilizing time of flight (TOF) principles. At step Error! Reference source not found., the position of the actinic dosage limit is dynamically adjusted based on the current distance “d” and a desired example 300 mm safety parameter “s”. The algorithm ensures that the line remains aligned with the safety parameter, continuously adapting to changes in the living organism's Error! Reference source not found. position. The safety parameter determines the distance at which the UV-C LED Error! Reference source not found. should be turned off to ensure the living organism Error! Reference source not found. remains within a safe exposure limit.

If the distance “d” is greater than the safety parameter “s,” step Error! Reference source not found. occurs, wherein the actinic dosage limit is moved further away from the UV-C LED Error! Reference source not found. to maintain the safe distance. The exact position adjustment can be calculated using a mathematical equation or algorithm specific to the system.

If the distance “d” is within or less than the safety parameter “s”, in step Error! Reference source not found. the actinic dosage limit remains at a position that guarantees the UV-C exposure is below the established safe limit. At Error! Reference source not found., the flow diagram restarts at Error! Reference source not found. to allow continuous monitoring. By continuously monitoring the living organism's Error! Reference source not found. distance and updating the actinic dosage limit accordingly, the processor ensures that the UV-C exposure remains within safe limits. The line's movement is responsive to the living organism's Error! Reference source not found. proximity, providing a safety buffer and accommodating changes in position. This dynamic adjustment guarantees that the disinfection process is at maximum effectiveness while prioritizing the safety of living organisms.

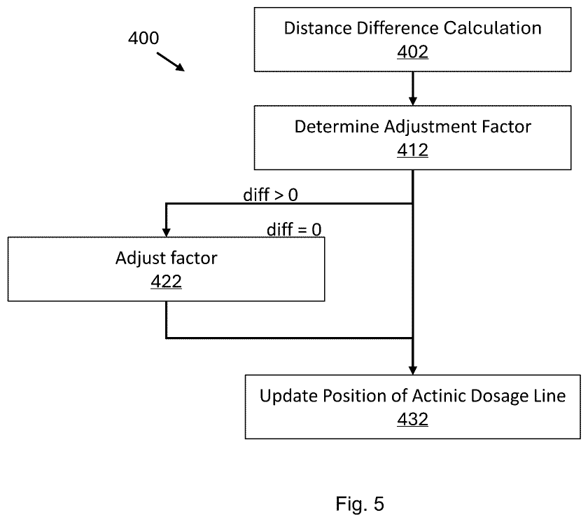

FIG. 5 illustrates a flow diagram as a general outline of one example for implementing the dynamic adjustment of the actinic dosage limit based on distance Error! Reference source not found. The specific equation and values used in the algorithm may vary depending on the system requirements and calibration process. At step Error! Reference source not found., A distance difference, “diff,” is calculated between the current distance “d” and the safety parameter “s” described above. The distance difference represents how far a living organism Error! Reference source not found. (FIG. 1) is from the desired safety parameter. If the living organism Error! Reference source not found. is closer than the safety parameter, the distance difference will be negative or zero, indicating that no adjustment is needed. If the living organism Error! Reference source not found. is farther away, the distance difference will be positive. The distance difference is calculated using the equation: diff=d−s. At step Error! Reference source not found., an adjustment factor “factor,” is determined based on the distance difference. The adjustment factor is calculated to linearly adjust the position of the actinic dosage limit. If the distance difference is greater than zero, step Error! Reference source not found. occurs, where the adjustment factor is calculated using the equation:

$$factor = \frac{diff}{d} .$$

If the distance difference is not greater than zero, then the factor is set to zero, as no adjustment is needed because the distance is already within or less than the safety parameter. At Error! Reference source not found., the position of the actinic dosage limit is updated using the equation: line_position=s+(factor*s). The line_position represents the updated position of the actinic dosage limit based on the safety parameter and the adjustment factor. By continuously recalculating the distance and updating the line_position, the algorithm ensures that the actinic dosage limit dynamically moves to maintain the desired safety parameter.

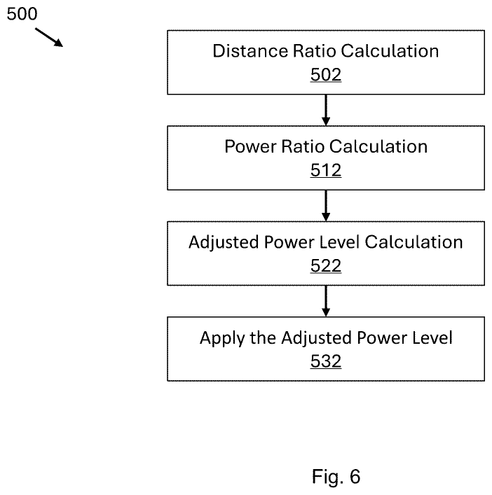

FIG. 6 illustrates an example flow diagram for an algorithm to dynamically adjust the power level for the UV-C LED Error! Reference source not found. At step Error! Reference source not found., the distance ratio, “ratio,” is calculated using the current distance “d” and the desired safety parameter “s” describe above. The distance ratio is calculated as the current distance divided by the desired safety parameter. The distance ratio represents how far the living organism Error! Reference source not found. (FIG. 1) is from the desired safety distance. The equation to calculate the distance ration can be expressed as:

$$ratio = \frac{d}{s} .$$

At step Error! Reference source not found., the power ratio, “power_ratio,” is calculated by scaling the distance ratio, mapping it to the range between “power_min” and “power_max”. This scaling allows for the adjustment of the power level based on the proximity of the living organism Error! Reference source not found. to the UV-C LED Error! Reference source not found. (FIG. 1). The variable “power_min” is the minimum power level for the UV-C LED Error! Reference source not found. and “power_max” is the maximum power level for the UV-C LED Error! Reference source not found. The equation to calculate the power ratio can be expressed as: power_ratio=ratio*(power_max-power_min). At step Error! Reference source not found., the adjusted power level is calculated. the adjusted power level “power_adjusted” is calculated by adding the power ratio to the minimum power level. This ensures that the power level remains within the specified range. The equation to calculate the adjusted power level can be expressed as power_adjusted=power_min+power_ratio. This equation assumes a linear relationship between the power level and distance. Depending on the specific characteristics of the UV-C LED Error! Reference source not found. module and the desired power adjustment behavior, alternative equations or algorithms can be implemented to suit the requirements. At step Error! Reference source not found., the adjusted power level is applied to the UV-C LED Error! Reference source not found. By applying this dynamic adjustment algorithm, the controller Error! Reference source not found. (FIG. 2) can calculate the optimal power level for the UV-C LED Error! Reference source not found. based on the current distance, desired safety parameter, and the predefined power limits. To summarize, the equation to calculate the adjusted

$$\begin{matrix}Power required would be : power\_adjusted = power\_min + ( \frac{d}{s} ) * \\ ( power\_max - power\_min )\end{matrix}$$

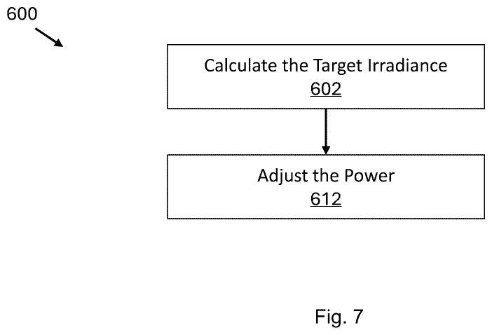

FIG. 7 illustrates a flow diagram exemplarily showing how to maintain a constant irradiance at the living organism's location as if they were at a safe distance Error! Reference source not found. The calculations required rely on applying the inverse square law, states that the intensity of light at a distance from a point source is inversely proportional to the square of that distance. It can be modeled by the equation

$$E = \frac{P}{4 π {d}^{2}} ,$$

where “E” is the irridance at distance “d”, “P” is the power output of the source (radiant flux), and “d” is the distance from the source to the target. At Error! Reference source not found., the target irradiance is calculated. A required power must be adjusted based on the square of the ratio of d to s. Thus, the target irradiance Eₛ can be expressed as

$${E}_{s} = \frac{Φ}{4 * π * {s}^{2}} ,$$

where Φ is the radiant flux. The radiant flux is the power emitted by the UV-C LED in milliwatts and varies depending on the UV-C LED Error! Reference source not found. (FIG. 1). The specific value can be located in the UV-C LED's Error! Reference source not found. specification. At Error! Reference source not found., the power is adjusted to maintain the target irradiance at a new distance. This calculation can be expressed as:

$$P = {E}_{s} * 4 π {d}^{2} = ( \frac{Φ}{4 π {s}^{2}} ) * 4 π {d}^{2} = Φ ( \frac{{d}^{2}}{{s}^{2}} ) .$$

The calculations described in FIG. 7 can be simplified down to

$$P = Φ {( \frac{d}{s} )}^{2} .$$

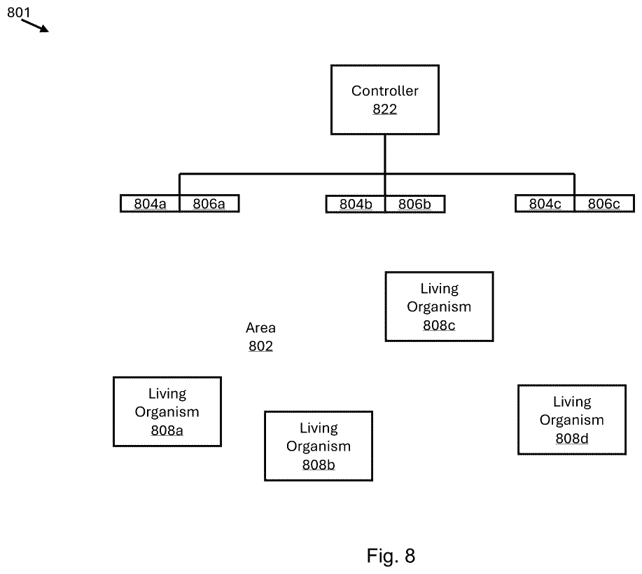

FIG. 8 shows a system 801 for controlling multiple UV-LEDs that are directed to an area 802 that contains multiple living organisms 808a, 808b, 808c, and 808d, representing any number of living organisms in the area 802. The area 802 is representative of any of a variety of areas large enough to require multiple UV-C sources to disinfect. Examples of such areas include airplanes, train cars, restaurants, conference/banquet rooms, theaters, and auditoriums, to give a few non-exclusive examples.

The illustrated system includes three UV-C LEDs 804a, 806b, and 806c, representative of multiple UV-C LEDs (of any number) that direct UV-C into the space 802. The UV-C LEDs 804a, 804b, and 804c may be located along with respective mmWave sensors 806a, 806b, and 806c.

A controller 822 is operatively coupled to the UV-C LEDs 804a, 804b, and 804c, and to the mmWave sensors 806a, 806b, and 806c, to control output from the UV-C LEDs 804a, 804b, and 804c (for instance), based at least in part on input from the mmWave sensors 806a, 806b, and 806c. The output from each of the UV-C LEDs 804a, 804b, and 804c may be individually set by the controller 822 based on their proximity to the living organisms 808a, 808b, 808c, and 808d, and in consideration of the output levels by the other of the UV-C LEDs 804a, 804b, and 804c. The controller 822 may be configured to set the output levels of the UV-C LEDs 804a, 804b, and 804c so as to provide adequate disinfection where the living organisms are located, without providing an excessive UV-C dose to any of the living organisms. When determining what values to configure the UV-C LEDs 804a, 804b, and 804c outputs at, the controller considers the full width at half maximum (FWHM) values of each UV-C LED 804a, 804b, and 804c. Using the FWHMs, the controller can determine any overlapping UV irradiance areas from the UV-C LEDs 804a, 804b, and 804c in the space 802 and adjust the individual values of each UV-C LED 804a, 804b, and 804c to ensure the living organism is not exposed to an excessive UV-C dose.

The controller 822 may be a separate device, or its functions may be performed at the location of one of the UV-C LEDs 804a, 804b, and 804c, and/or at one of the mmWave sensors 806a, 806b, and 806c. Alternatively, the functions of the controller 822 may be distributed over multiple connected devices, some of which may be co-located with the UV-C LEDs 804a, 804b, and 804c, and/or at one of the mmWave sensors 806a, 806b, and 806c. Wired and/or wireless communications methods may be used to connect (as in operatively couple) the various components of the system 801 together, such as in a network.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

### REFERENCE DESIGNATORS

1. System
2. Area
4. UV-C LED
6. mmWave Sensor
8. Living Organism
10. Area
122. Controller
142. Processor
152. Storage
162. Memory
172. Bus
200. Dynamic Adjustment Algorithm
202. Distance Calculation
204. Fail-safe Mechanism
212. Calculation of Proximity Ratio
222. Power Adjustment Calculation
232. Power Level Setting
300. Method of Moving an Actinic dosage limit
302. Distance Calculation
312. Updating the Actinic dosage limit
322. Actinic dosage limit is moved further away
332. Actinic dosage limit remains at a position
342. Repeat
400. Outline for Implementing the Dynamic Adjustment of the Actinic dosage limit
402. Distance Difference Calculation
412. Determine Adjustment Factor
422. Adjust factor
432. Update Position of Actinic dosage limit
500. Algorithm to Dynamically Adjust the Power Level for the UV-C LED
502. Distance Ratio Calculation
512. Calculate Power Ratio
522. Adjusted Power Level Calculation
532. Apply the Adjusted Power Level
600. Method to Maintain a Constant Irradiance
602. Calculate the Target Irradiance
612. Adjust the Power
801. System
802. Area
804a, 804b, and 804c. UV-C LEDS
806a, 806b, and 806c. mmWave sensors
808a, 808b, 808c, and 808d Living organisms
822. Controller

What is claimed is:

1. A system for controlling a UV-C LED, comprising:
a mmWave sensor configured to detect a living organism using a micro-Doppler effect and measure a distance between the sensor and the living organism; and
a controller programmed to receive the measured distance and calculate an adjusted power level for the UV-C LED based on the measured distance to maintain a predetermined safe UV-C irradiance level.

2. The system of claim 1, wherein the controller employs a dynamic adjustment algorithm to determine the adjusted power level of the UV-C LED to ensure that the UV-C irradiance remains below a maximum allowable exposure level as the living organism's distance changes.

3. The system of claim 2, wherein the dynamic adjustment algorithm calculates a proximity ratio based on the measured distance and a predefined safety parameter, and adjusts the power level of the UV-C LED as a function of the proximity ratio.

4. The system of claim 3, wherein the predefined safety parameter forms a safety threshold that when exceeded, the UV-C LED is deactivated to ensure safety.

5. The system of claim 1, further including a fail-safe mechanism that triggers a shutdown of the UV-C LED if an error is detected in distance measurements or power control processes.

6. The system of claim 1, wherein the controller uses pulse-width modulation (PWM) techniques to adjust the power supplied to the UV-C LED.

7. A method of controlling a UV-C disinfection system, comprising:
measuring a distance between a UV-C LED and a living organism, using a mmWave sensor;
calculating a power level for the UV-C LED that ensures the irradiance does not exceed safe exposure limits based on the measured distance;
adjusting the power of the UV-C LED according to the calculated power level.

8. The method of claim 7, wherein a predefined safe distance defines a safety threshold of safe exposure, and the system deactivates the UV-C LED if the living organism comes within this distance.

9. The method of claim 7, wherein the power adjustment continually adjusts the irradiance at the location of the living organism as if the living organism was at the predefined safe distance.

10. The method of claim 7, further comprising dynamically updating an actinic dosage limit based on the proximity of the living organism to the UV-C LED to maintain the desired safety margin.

11. The method of claim 7, further comprising employing a fail-safe mechanism that triggers if the calculated power level exceeds a safe threshold.

12. A UV-C LED control system configured to dynamically adjust irradiance levels based on real-time distance measurements, comprising:
a mmWave sensor for detecting the distance of a living organism from the UV-C LED;
a controller programmed to calculate a necessary adjustment in the UV-C LED power to maintain irradiance within safe exposure limits based on the distance measured by the mmWave sensor.

13. The system of claim 12, wherein the controller uses a mathematical algorithm that incorporates the inverse square law to adjust the UV-C LED power to ensure consistent irradiance levels at varying distances.

14. The system of claim 13, where the mathematical algorithm adjusts the power output of the UV-C LED based on the ratio of the current distance to a predefined safety distance.

15. The system of claim 12, wherein the mmWave sensor provides continuous real-time tracking movements of the living organism to dynamically adjust the UV-C exposure levels.

16. The system of claim 12, wherein the controller incorporates a fail-safe mechanism that is activated upon detection of any error in the distance measurement or power adjustment process.

17. The system of claim 12, further comprising a mechanism to dynamically update the position of an actinic dosage limit that moves in response to changes in the living organism's distance to the UV-C LED.

18. A system for controlling UV-C LEDs, comprising:
mmWave sensors configured to detect living organism(s) using a micro-Doppler effect and to measure distance(s) between the sensors and the living organism(s); and
a controller programmed to receive the measured distance(s), and to calculate adjusted power levels for the UV-C LEDs based on the measured distance(s), to maintain a predetermined safe UV-C irradiance level for the living organism(s).
